SEQUENCE LISTING (5 sequences)

SEQ ID NO: 1 - protein, 8 aa, Artificial
  Angiotensin receptor type 2 agonist
DRVYIHPF

SEQ ID NO: 2 - protein, 8 aa, Artificial
  Angiotensin receptor type 2 agonist
DRVYIXPF

SEQ ID NO: 3 - protein, 7 aa, Artificial
  Angiotensin receptor type 2 agonist
DRVYIHP

SEQ ID NO: 4 - protein, 6 aa, Artificial
  Angiotensin receptor type 2 agonist
RPLKPW

SEQ ID NO: 5 - protein, 8 aa, Artificial
  Angiotensin receptor type 2 agonist
XXXXXXXX

(19) United States
(12) Patent Application Publication
(10) Pub. No.: US 2015/0119329 A1
(43) Pub. Date: Apr. 30, 2015

(54) MODULATION OF ANGIOTENSIN II RECEPTORS FOR THE PREVENTION AND TREATMENT OF MALARIA CEREBRAL

(71) Applicants: Ana Maria Rodriguez Fernandez, Westbury, NY (US); Julio Gallego-Delgado, New York, NY (US)

(72) Inventors: Ana Maria Rodriguez Fernandez, Westbury, NY (US); Julio Gallego-Delgado, New York, NY (US)

(73) Assignee: NEW YORK UNIVERSITY, New York, NY (US)

(21) Appl. No.: 14/394,332

(22) PCT Filed: Apr. 16, 2013
(86) PCT No.: PCT/US2013/036763

§ 371 Date: Oct. 14, 2014

Related U.S. Application Data
(60) Provisional application No. 61/624,722, filed on Apr. 16, 2012.

(51) Int. Cl. A61K 38/07; A61K 31/41; A61K 31/416; A61K 45/06; A61K 31/4155

(52) U.S. Cl. CPC A61K 38/07; A61K 45/06; A61K 31/4155; A61K 31/416; A61K 31/41
USPC 514/13.3; 514/381; 514/406; 514/382; 514/364

(57) ABSTRACT

The present invention is directed to a composition for the treatment or prevention of cerebral malaria that comprises an angiotensin receptor type-2 agonist and an antimalaria drug. The present invention is further directed to methods for treating and preventing cerebral malaria that involve administering an angiotensin receptor type-2 agonist and/or an angiotensin receptor type-1 antagonist.

This application claims the benefit of U.S. Provisional Patent Application Ser. No. 61/624,722 filed Apr. 16, 2012, which is hereby incorporated by reference in its entirety.

### FIELD OF THE INVENTION

This invention relates to compositions and methods for the treatment and prevention of cerebral malaria

### BACKGROUND OF THE INVENTION

With approximately 216 million cases during 2010, malaria remains the most important parasitic disease of humans, resulting in more than 600,000 deaths every year, mostly in African children under the age of five. More than 25 million travelers from non-tropical regions visit malaria-endemic countries every year, and about 30,000 cases of travel-associated clinical malaria occur annually. Non-immune travelers are at risk for Plasmodium falciparum malaria and therefore, for developing cerebral malaria, during travel in malaria-endemic countries, which include most of Africa, South America and South Asia.

Cerebral malaria is the most profound syndrome of severe malaria characterized by impaired consciousness, generalized convulsions, coma and neurological sequelae. It can be fatal within 24-72 hours. The interaction between Plasmodium falciparum infected red blood cells (iRBCs) and host brain endothelial cells plays a key role in cerebral malaria. Specifically, mature stage parasites express ligands (PfEMP1) on the surface of iRBC that interact with host endothelial cell receptors (ICAM-1) leading to their sequestration into the brain microcirculation. Expression of ICAM-1 is increased by inflammatory mediators, enhancing the binding of iRBC. Sequestration of iRBC promotes the loss of endothelial cell junctions, endothelial apoptosis, and ultimately the disruption of the blood-brain barrier (BBB). This disruption causes a massive diffusion from the circulation into the brain tissue leading to coma and damage to the nervous system.

The present invention is directed to overcoming these and other limitations in the art.

### SUMMARY OF THE INVENTION

A first aspect of the present invention relates to a pharmaceutical composition for the treatment or prevention of cerebral malaria. This composition comprises an angiotensin receptor type-2 agonist and an antimalaria drug.

Another aspect of the present invention is directed to a method of preventing or treating cerebral malaria in a subject. This method involves selecting a subject having or at risk of having malaria and administering, to the selected subject, an angiotensin receptor type-2 agonist under conditions effective to prevent or treat cerebral malaria in the subject.

Another aspect of the present invention is directed to a method of preventing or treating cerebral malaria in a subject. This method involves selecting a subject having or at risk of having malaria and administering, to the selected subject, an angiotensin receptor type-1 antagonist under conditions effective to prevent or treat cerebral malaria in the subject.

While available anti-malarial drugs are effective at clearing parasites from the blood, they do not have specific effects against cerebral malaria. The only therapeutic strategy currently available is to administer high doses of conventional anti-malarials and hope that the parasite will be eliminated before cerebral malaria causes death of the patient. An effective, specific treatment for cerebral malaria would have a striking impact on the deaths caused by malaria, since this syndrome is responsible for the majority of the mortality attributed to P. falciparum infection. As disclosed herein, angiotensin receptor type-1 antagonists and angiotensin receptor type-2 agonists counteract infective red blood cell induced disruption of endothelial cell junctions, thereby maintaining the integrity of the blood brain barrier and preventing the manifestations of cerebral malaria.

### BRIEF DESCRIPTION OF THE DRAWINGS

FIGS. 1A-1F show human brain endothelial cells (HBECs) incubated with control uninfected erythrocytes (FIG. 1A); Plasmodium faciparum infected red blood cells (iRBC) (FIG. 1B); iRBC+Losartan (angiotensin receptor type-1 antagonist; FIG. 1C); and iRBC+CGP-42112A (angiotensin receptor type-2 agonist; FIG. 1D). Actin (red), Beta-catenin (green), DNA (blue). FIG. 1E is a graph showing endothelial cell survival as measured by quantification of endothelial cell nuclei by microscopy of no treatment control endothelial cells (control), endothelial cells incubated with iRBCs alone (iRBCs), or in the presence of Losartan (iRBC+Los) or CGP-42112A (iRBC+CPG). FIG. 1F is a graph also showing endothelial cell survival as measured by Presto Blue® fluorescence quantitation of no treatment control endothelial cells (control), endothelial cells incubated with iRBCs alone (iRBCs), or in the presence of Losartan (iRBC+Los) or CGP-42112A (iRBC+CPG).

FIG. 2 is a graph showing the effect of angiotensin receptor type-1 (ATI) antagonism (Losartan) and angiotensin receptor type-2 (AT2) agonism (CGP-42112A) on the development of cerebral malaria in an experimental in vivo mouse model.

FIGS. 3A-3C show hematoxilinleosin staining of brain tissue sections taken from mice having cerebral malaria. FIG. 3A is a tissue section taken from a control animal having cerebral malaria. The tissue sections shown in FIGS. 3B and 3C are from wildtype mice infected with Plasmodium berghei ANKA and treated with Losartan (ATI antagonist) and CGP-42112A (AT2 agonist), respectively. Disruption of the blood brain barrier is evidenced by the presence of hemorrhages (arrows) shown in FIG. 3A.

FIG. 4 is a bar graph quantifying the number of hemorrhages in brain histological slices taken from control animals having cerebral malaria, and animals infected with P. berghei ANKA and treated with Losartan (ATI antagonist) and CGP-42112A (AT2 agonist), respectively.

### DETAILED DESCRIPTION OF THE INVENTION

A first aspect of the present invention relates to a pharmaceutical composition for the treatment or prevention of cerebral malaria. This composition comprises an angiotensin receptor type-2 agonist and an antimalaria drug.

In accordance with this aspect of the present invention, the angiotensin receptor type-2 agonist is any agent that increases activity of the angiotensin type-2 receptor. Suitable agonists include angiotensin type-2 receptor peptide and non-peptide agonists.

An exemplary angiotensin type-2 receptor peptide ligand includes angiotensin II, which comprises an amino acid sequence of Asp-Arg-Val-Tyr-Ile-His-Pro-Phe (SEQ ID NO: 1). Variants, analogues and mimetics of the angiotensin II are also suitable for use in the composition and methods of the present invention. Suitable peptide mimetics include, for example and without limitation, p-aminophenylalanine-angiotensin II (Asp-Arg-Val-Tyr-Ile-p-Amino-Phe-Pro-Phe; SEQ ID NO: 2) and angiotensin (1-7) heptapeptide (Asp-Arg-Val-Tyr-Ile-His-Pro; SEQ ID NO: 3).

A suitable angiotensin type-2 receptor peptide agonist for use in the composition and methods of the present invention includes CGP-42112A (IUPAC name: (25,35)-2-[[(25)-1-[(25)-2-[[(25)-6-[[(25)-5-(diaminomethylideneamino)-2-(phenylmethoxy-carbonylamino)pentanoyl]amino]-2-[[(25)-3-(4-hydroxyphenyl)-2-(pyridine-3-carbonylamino)propanoyl]amino]hexanoyl]amino]-3-(1H-imidazol-5-yl)propanoyl]pyrrolidine-2-carbonyl]amino]-3-methylpentanoic acid) described in WO1999/43339 to Fändriks et al., which is hereby incorporated by reference in its entirety. The structural formula of CGP-42112A is depicted below:

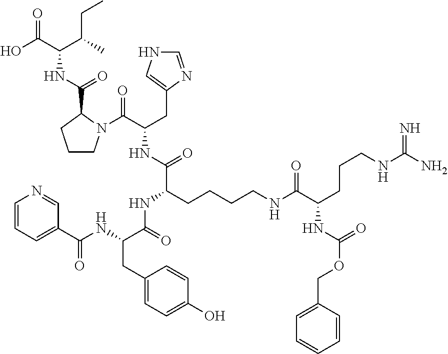 [SMILES: CC[C@H](C)[C@H](NC(=O)[C@@H]1CCCN1C(=O)[C@H](Cc1c[nH]cn1)NC(=O)[C@H](CCCCNC(=O)[C@H](CCCNC(=N)N)NC(=O)OCc1ccccc1)NC(=O)[C@H](Cc1ccc(O)cc1)NC(=O)c1cccnc1)C(=O)O]

It will be appreciated by those skilled in the art that the structure of CGP-42112A may be substituted or otherwise modified in one or more positions, as long as this substitution or modification does not significantly affect the agonistic effect of the compound and does not result in a derivative that is pharmacologically unacceptable due to toxicity issues. For example, one or more hydrogens of the C—H bonds of the heterocyclic ring system may be replaced by halogen, e.g. chlorine, bromine or iodine atoms. Further, the hydrogens of the C—H bonds may also be replaced by alkyl groups, e.g. methyl, ethyl or propyl groups.

Another suitable angiotensin type-2 receptor peptide agonist for use in the composition and methods of the present invention includes Novokinin (Arg-Pro-Leu-Lys-Pro-Trp; SEQ ID NO: 4), described by Yamada et al., “A Potent Hypotensive Peptide, Novokinin, Induces Relaxation by AT2- and IP-receptor Dependent Mechanism in the Mesenteric Artery from SHRs,” Biosci. Biotechnol. Biochem. 72:257 (2008), which is hereby incorporated by reference in its entirety.

Other suitable angiotensin type-2 receptor peptide agonists include those having the amino acid sequence of R¹—R²—R³—R⁴—R⁵—R⁶—R⁷—R⁸ (SEQ ID NO: 5) (U.S. Pat. No. 5,834,432 to Rodgers et al., which is hereby incorporated by reference in its entirety)

wherein R¹ and R² together form a group of formula X—R^{A}—R^{B}

wherein X is H or a one to three peptide group, and a peptide bond between R^{A} and R^{B} is labile to aminopeptidase A cleavage;

R^{A} is selected from the group consisting of Asp, Glu, Asn, 1-aminocyclopentane carboxylic acid (Acpc), Ala, N,N-dimethylglycyl, Pro, 1-carboxy-N,N,N-trimethylmethanaminium hydroxide inner salt (betaine), Glu(NH₂), Gly, Asp(NH₂) and succinyl;

R^{B} is selected from the group consisting of Arg, Lys, Ala, Ornithine (Orn), Ser(Ac), N-methylglycyl (sarcosine), D-Arg and D-Lys.
R³ is selected from the group consisting of Val, Ala, Leu, Ile, Gly, Pro, 2-aminoisobutyric acid, Acpc and Tyr;
R⁴ is selected from the group consisting of Tyr, Thr, Ser and aza-α′-homo-L-tyrosyl;
R⁵ is selected from the group consisting of Ile, Ala, Leu, Val and Gly;
R⁶ is p-NH₂-Phe;
R⁷ is Pro or Ala; and
R⁸ is selected from the group consisting of Phe, p-bromo-L-phenylalanyl, Ile and Tyr.
In some embodiments, preferred combinations of R^{A} and R^{B} include, without limitation, Asp-Arg, Asp-Lys, Glu-Arg and Glu-Lys.
In the above formulas, the standard three-letter abbreviations for amino acid residues are employed. In the absence of an indication to the contrary, the L-form of the amino acid is intended

Suitable non-peptide angiotensin type-2 receptor agonists for use in the composition and methods of the present invention include the tricyclic agonists disclosed in U.S. Pat. No. 7,652,054 to Alterman et al., which is hereby incorporated by reference in its entirety. These non-peptide angiotensin type-2 receptor agonists have a formula of formula I as shown below,

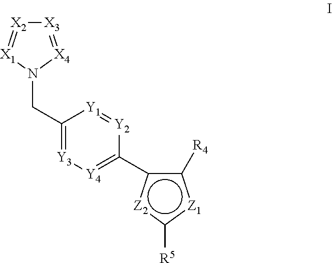

wherein
one of X₁ and X₂ represents —N— and the other represents —C(R¹)—;
X₃ represents —N— or —C(R²)—;
X₄ represents —N— or —C(R³)—;
R¹, R² and R³ independently represent H, C₁₋₆ alkyl, C₁₋₆ alkoxy, C₁₋₆ alkoxy-C₁₋₆-alkyl or halo;
provided that, when X₁ represents —C(R¹)—, X₃ represents —C(R²)—, and X₄ represents —C(R³)—, then R¹ represents H;
Y₁, Y₂, Y₃ and Y₄ independently represent —CH— or —CF—;
Z₁ represents —CH—, —O—, —S—, —N— or —CH═CH—;
Z₂ represents —CH—, —O—, —S— or —N—;
provided that:
(a) Z₁ and Z₂ are not the same;
(b) when Z₁ represents —CH═CH—, then Z₂ may only represent —CH— or —N—; and
(c) other than in the specific case in which Z₁ represents —CH═CH—, and Z₂ represents —CH—, when one of Z₁ and Z₂ represents —CH—, then the other represents —O— or —S—;
R⁴ represents —S(O)₂N(H)C(O)R⁶, —S(O)₂N(H)S(O)₂R⁶, —C(O)N(H)S(O)₂R⁶, or, when Z₁ represents —CH═CH—, R⁴ may represent —N(H)S(O)₂N(H)C(O)R⁷ or —N(H)C(O)N(H)S(O)₂R⁷;
R⁵ represents C₁₋₆ alkyl, C₁₋₆ alkoxy, C₁₋₆ alkoxy-C₁₋₆-alkyl, or di-C₁₋₃-alkylamino-C₁₋₄-alkyl;
R⁶ represents C₁₋₆ alkyl, C₁₋₆alkoxy, C₁₋₆ alkoxy-C₁₋₆-alkyl, C₁₋₃ alkoxy-C₁₋₆-alkoxy, C₁₋₆ alkylamino or di-C₁₋₆ alkylamino; and
R⁷ represents C₁₋₆ alkyl,
or a pharmaceutically-acceptable salt thereof.

An exemplary compound of Formula I comprises butyl (3-(4-((1H-imidazol-1-yl)methyl)phenyl)-5-isobutylthiophen-2-yl)sulfonylcarbamate, also known as Compound 21 (see Wan et al., “Design, Synthesis, and Biological Evaluation of the First Selective Nonpeptide AT2 Receptor Agonist,” J. Med. Chem. 47(24):5995-6008 (2004), which is hereby incorporated by reference in its entirety).

Other suitable angiotensin type-2 receptor non-peptide tricyclic agonists for use in the composition and methods of the present invention include those disclosed in U.S. Pat. No. 8,067,418 to Alterman et al., WO/2003/064414 to Hallberg et al., WO/2006/109048 to Alterman et al., WO/2004/046137 to Alterman et al., and WO/2004/046141 to Alterman et al., which are hereby incorporated by reference in their entirety. Suitable angiotensin type-2 receptor non-peptide bicyclic agonists for use in the composition and methods of the present invention include those disclosed in WO/2004/085420 to Hallberg et al., WO/2004/046128 to Hallberg et al., and WO/2006/109058 to Alterman et al., which are hereby incorporated by reference in their entirety.

The composition of the present invention may contain any one or more of the aforementioned angiotensin receptor type-2 agonists in combination with one or more antimalaria drugs. Suitable antimalaria drugs of the composition include any antimalaria drug known in the art including, without limitation, quinine and derivatives thereof, chloroquine, amodiaquine, pyrimethamine, proguanil, sulfonamide, mefloquine, atovaquone, primaquine, artemisinin, artemether, artesunate, arteether, halofantrine, doxycycline, clindamycin, and combinations thereof. Pharmacologically acceptable salts, hydrates, or solvates of the aforementioned anti-malaria drugs are also suitable for use in the compositions of the present invention.

In one embodiment of the present invention, the composition further comprises an angiotensin receptor type-1 antagonist. Suitable angiotensin receptor type-1 antagonists include, without limitation, Losartan ((2-butyl-4-chloro-1-{[2′-(1H-tetrazol-5-yl)biphenyl-4-yl]methyl]-1H-imidazol-5-yl)methanol) (see U.S. Pat. No. 5,138,069 to Carini et al., and U.S. Pat. No. 5,608,075 to Campbell et al., which are hereby incorporated by reference in their entirety), Candesartan (2-ethoxy-1-({4-[2-(2H-1,2,3,4-tetrazol-5-yl)phenyl]phenyl}methyl)-1H-1,3-benzodiazole-7-carboxylic acid) (see U.S. Pat. No. 7,692,023 to Etinger et al., which is hereby incorporated by reference in its entirety), Valsartan ((S)-3-methyl-2-(N-{[2′-(2H-1,2,3,4-tetrazol-5-yl)biphenyl-4-yl]methyl}pentanamido)butanoic acid) (see U.S. Pat. No. 5,399,578 to Buhlmayer et al., which is hereby incorporated by reference in its entirety), Irbesartan (2-butyl-3-({4-[2-(2H-1,2,3,4-tetrazol-5-yl)phenyl]phenyl}methyl)-1,3-diazaspiro[4.4]non-1-en-4-one), Telmisartan (2-(4-{[4-methyl-6-(1-methyl-1H-1,3-benzodiazol-2-yl)-2-propyl-1H-1,3-benzodiazol-1-yl]methyl}phenyl)benzoic acid), Eprosartan (4-({2-butyl-5-[2-carboxy-2-(thiophen-2-ylmethyl)eth-1-en-1-yl]-1H-imidazol-1-yl}methyl)benzoic acid), Olmesartan ((5-methyl-2-oxo-2H-1,3-dioxol-4-yl)methyl4-(2-hydroxypropan-2-yl)-2-propyl-1-({4-[2-(2H-1,2,3,4-tetrazol-5-yl)phenyl]phenyl}methyl)-1H-imidazole-5-carboxylate), Azilsartan ((5-methyl-2-oxo-1,3-dioxol-4-yl)methyl 2-ethoxy-1-([2′-(5-oxo-4,5-dihydro-1,2,4-oxadiazol-3-yl)biphenyl-4-yl]methyl)-1H-benzimidazole-7-carboxylate), EXP3174 (Losartan acid metabolite), and combinations thereof.

Compositions of the present invention may further comprises one or more additional active agents including for example, a diuretic (e.g., mannitol and urea), an anti-convulsant (e.g., diazepam, phenyloin, phenobarbital, and phenobarbitone), an anti-pyretic (e.g., paracetamol), an anti-oxidant, and an anti-inflammatory drug (e.g., NSAIDS, steroids, cyclosporin, thalidomide, revlimid, anti-TNF antibodies (e.g., infliximab, etanercept), and pentoxifylline).

Compositions of the present invention may further comprise a pharmaceutically acceptable carrier. The phrase “pharmaceutically acceptable carrier” is art-recognized, and includes, for example, pharmaceutically acceptable materials, compositions or vehicles, such as a liquid or solid filler, diluent, solvent or encapsulating material, involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; cocoa butter and suppository waxes; oils, such as peanut oil, cottonseed oil, sunflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Selection of a suitable pharmaceutical carrier may depend on the route of administration. In accordance with this aspect of the present invention, the composition can be formulated for oral, rectal, intravenous, intramuscular, intraperitoneal, intranasal (e.g., by nasogastric tube), parenteral, topical, subcutaneous, intra-arterial, intracranial, or intradermal administration.

For example, for purposes of intraperitoneal administration, the angiotensin receptor type-2 agonist and anti-malarial drug may be incorporated into a solution or a suspension. The amount of angiotensin receptor type-2 agonist and anti-malarial drug which promotes P. falciparum clearance and prevents damage to the blood brain barrier in such compositions is such that a suitable dosage will be obtained. The solution or suspension may also include the following components: a sterile diluent, such as water for injection, saline solution, fixed oils, polyethylene glycols, glycerine, propylene glycol or other synthetic solvents: antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity or osmolarity, such as sodium chloride or dextrose. The preparation may be enclosed in ampoules, disposable syringes or multiple dose vials made of glass or plastic.

In some embodiments, the compositions are formulated for intravenous administration. The compositions may comprise the angiotensin receptor type-2 agonist and anti-malaria drug at a weight ratio in the range of about 1:7 to about 1:1500.

The composition of the present invention may be formulated for oral administration. For example, an angiotensin receptor type-2 agonist and anti-malaria drug may be formulated with an inert diluent, typically an edible carrier. They may be enclosed in gelatin capsules or compressed into tablets. For the purpose of oral therapeutic administration, the compounds may be incorporated with excipients and used in the form of tablets, troches, capsules, elixirs, suspensions, syrups, waters, chewing gums, and the like. The amount of the compounds employed in various embodiments of the present invention will be such that a suitable dosage will be provided in the administered amount. For oral administration the compositions may comprise the angiotensin receptor type-2 agonist and anti-malaria drug at a weight ratio in the range of about 1:1 to about 1:75.

Tablets, pills, capsules, troches and the like may contain the following ingredients: a binder, such as micro-crystalline cellulose, gum tragacanth or gelatin;

an excipient, such as starch or lactose; a disintegrating agent, such as alginic acid, Primogel, corn starch and the like; a lubricant, such as magnesium stearate or Sterotes; a glidant, such as colloidal silicon dioxide; a sweetening agent, such as sucrose, saccharin or aspartame; or flavoring agent, such as peppermint, methyl salicylate or orange flavoring.

When the dosage unit form is a capsule it may contain, in addition to compounds comprising embodiments of the present invention, a liquid carrier, such as a fatty oil. Other dosage unit forms may contain other materials that modify the physical form of the dosage unit, for example, as coatings. The coating(s) can be formulated for immediate release, delayed/enteric release or sustained release of the second pharmaceutical active in accordance with methods well known in the art. For example, a coating for immediate release is commonly used as a moisture barrier, and for taste and odor masking. Rapid breakdown of the coating in gastric media will lead to effective disintegration and dissolution. Thus, tablets or pills may be coated with sugar, shellac or other enteric coating agents. A syrup may contain, in addition to the active compounds, sucrose as a sweetening agent and preservatives, dyes, colorings and flavors. Materials used in preparing these compositions should be pharmaceutically pure and non-toxic in the amounts used.

The dosage form of the present invention may be either immediate or controlled release. For example, an immediate release form may comprise one or more pharmaceutically acceptable excipients including, but not limited to, one or more of microcrystalline cellulose, hydroxypropylcellulose, starch, lactose monohydrate, anhydrous lactose, talc, colloidal silicon dioxide, providone, citric acid, poloxamer, sodium starch glycolate, stearic acid, and magnesium stearate.

Controlled release can be achieved in the formulations by several mechanisms known in the art. For example, pH sensitive polymer or co-polymer can be used which when applied around the drug matrix functions as an effective barrier to release of the active agent at certain pH range. An alternative to a pH sensitive polymer or co-polymer is a polymer or co-polymer that is non-aqueous-soluble. The extent of resistance to release, for example, in the gastric environment can be controlled by coating with a blend of the non-aqueous-soluble and a aqueous soluble polymer. In this approach neither of the blended polymers or co-polymers are pH sensitive. One example of a pH sensitive co-polymer is the Eudragit® methacrylic co-polymers, including Eudragit® L 100, S 100 or L 100-55 solids, L 30 D-55 or FS 30D dispersions, or the L 12,5 or S 12,5 organic solutions. The polymers may be applied to a tablet for example, by spray coating (as a thin film), by compression coating or other suitable procedures. Polymer(s) may be applied over the surface of the capsule or applied to microparticles of the drug, which may then be encapsulated such as in a capsule or gel.

A sustained release film coat may be used for the compositions of the present invention including a water insoluble material such as a wax or a wax-like substance, fatty alcohols, shellac, zein, hydrogenated vegetable oils, water insoluble celluloses, polymers of acrylic and/or methacrylic acid, and any other slowly digestible or dispersible solids known in the art.

Another aspect of the present invention is directed to a method of preventing or treating cerebral malaria in a subject. This method involves selecting a subject having or at risk of having malaria, and administering, to the selected subject, an angiotensin receptor type-2 agonist under conditions effective to prevent or treat cerebral malaria in the subject.

Angiotensin receptor type-2 agonists suitable for administration in accordance with this aspect of the present invention are described supra.

In one embodiment of the present invention, the angiotensin receptor type-2 agonist is administered prophylactically to prevent, delay, or inhibit the development of cerebral malaria in a subject having malaria. In some embodiments of the present invention, prophylactic administration of an angiotensin receptor type-2 agonist is effective to fully prevent the development of cerebral malaria in an individual. In other embodiments, prophylactic administration is effective to prevent the full extent of cerebral malaria that would otherwise develop in the absence of such administration, i.e., substantially prevent or inhibit the development and/or severity of cerebral malaria in an individual.

In another embodiment of the present invention, the angiotensin receptor type-2 agonist is administered therapeutically to an individual having cerebral malaria to inhibit further development of the syndrome, i.e., to prevent ongoing damage to neurovascular endothelial cells and alleviate one or more symptoms of the condition.

In one embodiment of this aspect of the present invention, an angiotensin receptor type-1 antagonist is also administered to the selected subject in conjunction with the angiotensin receptor type-2 agonist. Suitable angiotensin receptor type-1 antagonists are described supra. The angiotensin receptor type-1 antagonist can be administered simultaneously or sequentially with the angiotensin receptor type-2 agonist. Administering can be repeated as necessary to prevent and/or treat cerebral malaria in the subject.

In another embodiment of this aspect of the present invention, one or more antimalaria drugs is administered to the selected subject in conjunction with the angiotensin receptor type-2 agonist. Suitable antimalaria drugs are described supra. The one or more antimalaria drugs can be administered simultaneously or sequentially with the angiotensin receptor type-2 agonist. Administering can be repeated as necessary to prevent and/or treat cerebral malaria in the subject.

In another embodiment of this aspect of the present invention, the angiotensin receptor type-2 agonist is administered in conjunction with one or more other therapeutic agents that are suitable for treating cerebral malaria. Suitable cerebral malaria therapeutic agents include, without limitation, α7nAChR agonists as disclosed in U.S. Patent Application Publication No. 20090239901 to Bencherif, which is hereby incorporated by reference in its entirety, Iloprost (5-{(E)-(1S,5S,6R,7R)-7-hydroxy-6[(E)-(3S, 4RS)-3-hydroxy-4-methyl-1-octen-6-inyl]-bi-cyclo[3.3.0]octan-3-ylidene}pentanoic acid) as disclosed in U.S. Pat. No. 5,466,713 to Blitstein-Willinger et al., which is hereby incorporated by reference in its entirety, S1P receptor agonists as disclosed in U.S. Patent Application Publication No. 20100112037 to Bachrach et al., which is hereby incorporated by reference in its entirety, and castanospermine esters as disclosed in U.S. Pat. No. 5,214,050 to Bitonti et al., which is hereby incorporated by reference in its entirety.

Another aspect of the present invention is directed to a method of preventing or treating cerebral malaria in a subject. This method involves selecting a subject having or at risk of having malaria and administering, to the selected subject, an angiotensin receptor type-1 antagonist under conditions effective to prevent or treat cerebral malaria in the subject.

Angiotensin receptor type-1 antagonists suitable for administration in accordance with this aspect of the present invention are described supra.

In one embodiment of the present invention, the angiotensin receptor type-1 antagonist is administered prophylactically to prevent, delay, or inhibit the development of cerebral malaria in a subject having malaria. In some embodiments of the present invention, prophylactic administration of an angiotensin receptor type-1 antagonist is effective to fully prevent the development of cerebral malaria in an individual. In other embodiments, prophylactic administration is effective to prevent the full extent of cerebral malaria that would otherwise develop in the absence of such administration, i.e., substantially prevent or inhibit the development and/or severity of cerebral malaria in an individual.

In another embodiment of the present invention, the angiotensin receptor type-1 antagonist is administered therapeutically to an individual having cerebral malaria to inhibit further development of the syndrome, i.e., to prevent ongoing damage to neurovascular endothelial cells and alleviate one or more symptoms of the condition.

In one embodiment of this aspect of the present invention an angiotensin receptor type-2 agonist and/or one or more antimalaria drugs are administered to the selected subject in conjunction with the angiotensin receptor type-1 antagonist. Suitable angiotensin receptor type-2 agonists and antimalaria drugs are described supra. The angiotensin receptor type-2 agonist and antimalaria drug can be administered simultaneously or sequentially with the angiotensin receptor type-1 antagonist. Administering can be repeated as necessary to prevent and/or treat cerebral malaria in the subject.

In another embodiment of this aspect of the present invention, the angiotensin receptor type-1 antagonist is administered in conjunction with one or more other therapeutic agents that are suitable for treating cerebral malaria as described supra.

For purposes of this and all aspects of the invention, the target “subject” encompasses any animal, preferably a mammal. Exemplary mammalian subjects include, without limitation, humans, non-human primates, dogs, cats, rodents (e.g., mouse, rat, guinea pig), horses, cattle and cows, sheep, pigs, and birds. In preferred embodiments of the present invention the subject is a human.

In the context of administering a composition of the invention for purposes of preventing cerebral malaria in a subject, the target subject encompasses any subject that is at risk for developing cerebral malaria, including subjects having P. falciparum on blood smear and optionally exhibiting one or more additional symptoms of malaria including those of severe malaria (e.g., severe malarial anemia, respiratory distress, shock, spontaneous bleeding, hypoglycemia, repeated seizures, hemoglobinuria, hypoglycemia, prostration, impaired consciousness, jaundice, hyperparasitemia). In the context of administering a composition of the invention for purposes of treating cerebral malaria in a subject, the target subject encompasses any subject infected with P. falciparum that exhibits one or more symptoms of cerebral malaria (e.g., coma with P. falciparum on blood smear, and no other known cause for coma).

In the context of using therapeutic compositions of the present invention to prevent cerebral malaria, a prophylactically effective amount of a composition comprising an angiotensin receptor type-2 agonist alone. an angiotensin receptor type-1 antagonist alone, or a combination of an angiotensin receptor type-2 agonist and angiotensin receptor type-1 antagonist is that amount capable of preventing one or more symptoms associated with cerebral malaria, decreasing the severity of at least one symptom, and preferably preventing all neurological complications in the subject.

In the context of using therapeutic compositions of the present invention to treat cerebral malaria, a therapeutically effective amount of a composition comprising an angiotensin receptor type-2 agonist alone, an angiotensin receptor type-1 antagonist alone, or a combination of an angiotensin receptor type-2 agonist and angiotensin receptor type-1 antagonist is that amount capable of achieving a reduction in symptoms associated with cerebral malaria, a decrease in the severity of at least one symptom, and preferably alleviating all neurological complications in the subject.

Therapeutically effective amounts of an angiotensin receptor type-2 agonist or angiotensin receptor type-1 antagonist composition can be determined in accordance with standard procedures, which take numerous factors into account, including, for example, the concentrations of these active agents in the composition, the mode and frequency of administration, the severity of cerebral malaria to be treated (or prevented), and subject details, such as age, weight, sex, other drugs being administered, and overall health and immune condition. Suitable dosages are in the range of about 0.01-100 mg/kg. General guidance can be found, for example, in the publications of the International Conference on Harmonization and in REMINGTON'S PHARMACEUTICAL SCIENCES (Mack Publishing Company 1990), which are hereby incorporated by reference in their entirety. A clinician may administer a composition containing an angiotensin receptor type-2 agonist and/or angiotensin receptor type-1 antagonist until a dosage is reached that provides the desired or required prophylactic or therapeutic effect. The progress of this therapy can be easily monitored by conventional assays. Variations in the needed dosage are to be expected in view of the variety of compounds available and the different efficiencies of various routes of administration. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery.

Therapeutically effective amounts of one or more anti-malaria drugs to be administered in conjunction with the angiotensin receptor type-2 agonist or angiotensin receptor type-1 antagonist composition of the present invention are known in the art, ranging from about 10 mg/dose to about 1500 mg/dose or about 1 mg/Kg/hour to about 10 mg/Kg/hour.

Therapeutic compositions of the present invention may be administered in a single dose, or in accordance with a multi-dosing protocol. For example, doses of the therapeutic composition can be administered hourly, daily, or weekly. Different dosages, timing of dosages and relative amounts of the therapeutic composition can be selected and adjusted by one of ordinary skill in the art. Modes of administration of the therapeutic compositions of the present invention are described supra.

Compositions of the present invention can be formulated for pharmaceutical use as described supra and administered by parenteral, topical, intravenous, oral, rectal, subcutaneous, intraperitoneal, intranasal, intra-arterial, intracranial, intradermal or intramuscular means for prophylactic and/or therapeutic treatment.

### EXAMPLES

The following examples are provided to illustrate embodiments of the present invention but are by no means intended to limit its scope.

### Materials and Methods for Example 1

Inmortalization of HBMECs cell line. Purified human brain microvascular endothelial cells (HBMEC) were purchased frozen from ScienCell Research Laboratories in passage one, thawed, seeded on culture flasks and grown to confluence in DME/F12 medium (pH 7.4) supplemented with 10% fetal bovine serum, 30 mg/ml endothelial cell growth supplement, and 10 mg/ml penicillin/streptomycin. Cells were incubated at 37° C. in 5% CO₂ in water-saturated air. Primary HBMEC in their second passage from isolation were infected with Lenti-SV40-Ta virus immortalization kit from Capital Biosciences. Briefly, cells were infected overnight in a 6 well plate with 2 mL/well supernatant in the presence of 8 μg/mL polybrene. After 18 hours, the viral supernatant was discarded and fresh culture medium was added to the cells before a 72 h step incubation at 37° C. Cells were then trypsinized and sub-cultured in two 12 well plates for 7 days. Monolayers were then observed for growth and morphology changes before being analyzed for the expression of SV40 large T antigen by enzyme-linked immunosorbent assay. The HBEC-4A clone was selected for our analyses.

Cell viability assay. To assess the viability of the adherent HBMECs PrestoBlue Cell Viability Reagent (Invitrogen) was utilized. Briefly, HBMECs were cultured in 96-well plates to confluence (5.5×10⁴ cells/mL) and experiments were performed using 4×10⁶ iRBCs/cm² with or without Angiotensin II (5×10⁻⁷ M), Losartan (10⁻⁵ M) and CGP-42112A (10⁻⁵ M) (n=6 per condition). After 18 hours, the plates were washed twice with PBS, and PrestoBlue Cell Viability Reagent diluted 1:10 with medium, was added. Samples were then incubated for 3 hours at 37° C. PrestoBlue is reduced from blue resazurin to red resorufin in the presence of viable cells. The absorbance (595 nm) was read with a Victor X3 plate reader.

Histopathological studies. In order to analyze the presence of hemorrhages in the brain, paraffin-embedded transverse sections of brain (4 μm thick) were stained with Hematoxillin/Eosin. Cerebral malaria was evaluated by assessing the presence or absence of hemorraghes in the total area.

Immunofluorescence microscopy. HBMECs were seeded on gelatin-coated glass coverslips at 5.5×10⁴ cells/mL density. Coverslips were fixed with 4% paraformaldehyde for 10 min at RT, followed by permeabilization with 0.5% Triton X-100. Non specific binding sites were blocked with blocking buffer (PBS 2 mg/mL BSA) for 1 hour at RT. Primary antibodies were diluted 1/400 in blocking buffer and incubated overnight at 4° C. Alexa-488-labeled or Alexa-546-labeled secondary antibody diluted 1/500 in blocking buffer were incubated for 1 h at RT. Phalloidine-Rhodamine-labeled or Phalloidine-488-labeled were diluted 1/2000 and incubated for 10 minutes at RT.

DAPI diluted 1 μg/mL was incubated for 5 minutes at RT. Primary antibodies for the detection of β-catenin, Phospho-GSK-3β (Ser9), GSK-3β were purchased from Cell Signaling. Vinculin and ZO-1 antibodies are from Invitrogen. All images were acquired and analyzed using MetaMorph Advanced v7.6.5.0 on a Olympus IX70 inverted microscope.

Cerebral Malaria Experimental Mouse Model. Female C57BL/6J mice were obtained from Taconic Farms Inc. All mice were maintained under barrier conditions. Water and normal laboratory diet were available ad libitum. The infection was performed by inoculating i.p. 10⁶ infected erythrocytes with P. berghei ANKA. All procedures involving animals conformed to the Guide for the Care and Use of Laboratory Animals published by US National Institutes of Health (NIH Publication No. 85-23, revised 1985) and were performed with the approval of the the Animal Care and Use Committee at the University of New York.

Alzet osmotic minipumps (Model 1002; ALZA Scientific Products, Mountain View, Calif., USA) were implanted into C57BL/6J mice at 4 months of age. Pumps were filled either with saline vehicle or solution of CGP-42112A (Sigma Chemical Co.) that delivered (subcutaneously) at 500 ng/min/kg for the duration of the study. Pumps were placed into the subcutaneous space of subcutaneous bupivacaine anesthetized mice through a small incision on the animal back slightly posterior of the shoulder blades that was closed with Reflex 7 mm wound closure system (ALZA Scientific Products, Mountain View, Calif., USA). All incision sites healed rapidly without any further complication. Losartan was administered in the drinking water and adjusted to a final dose of 10 mg/Kg/d

### Example 1

### Angiotensin II Receptor Modulation Reduces Cerebral Malaria

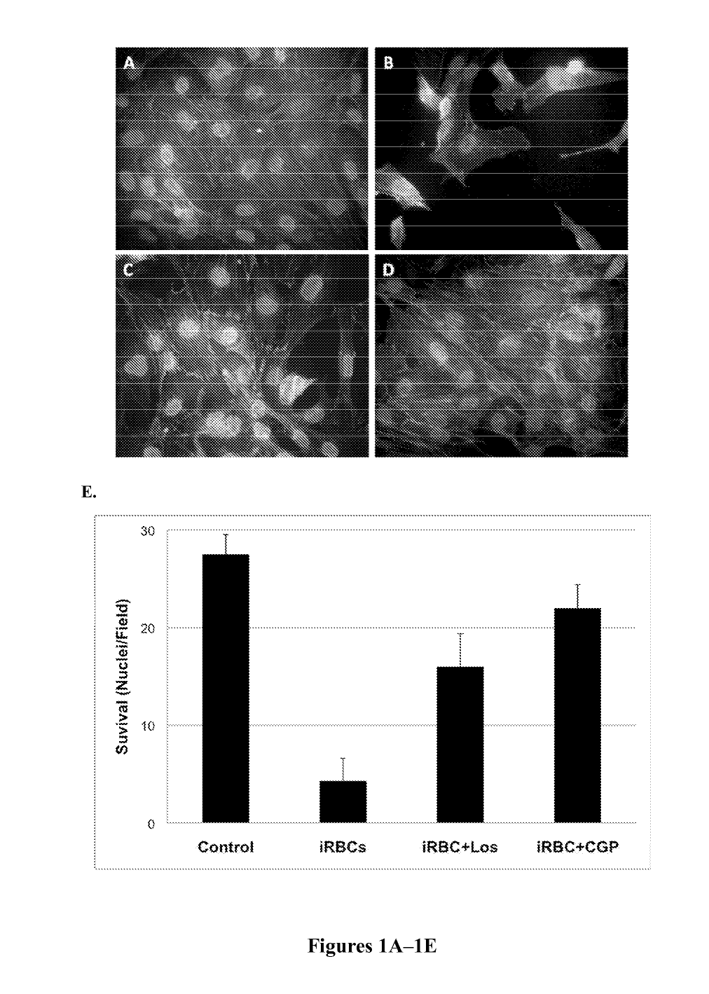

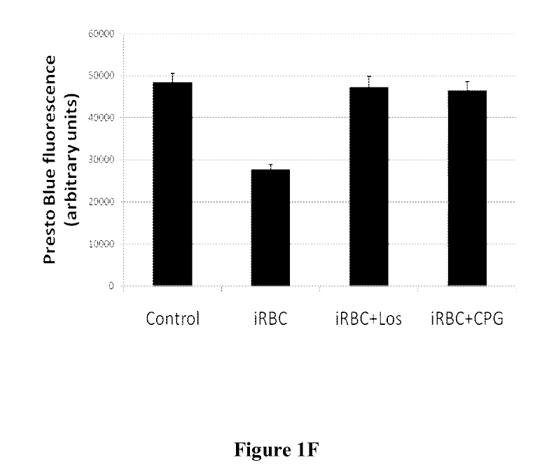

In an in vitro mouse model of cerebral malaria, rupture of P. falciparum infected red blood cells (iRBCs) causing the release of their contents over a monolayer of human brain microvascular endothelial cells (HBMECs) in vitro disrupts the interendothelial junctions, producing a contractile response, detachment and resulting in the opening of intercellular gaps (FIGS. 1A and 1B).

Interendothelial junctions are regulated by angiotensin II, the main vasoactive peptide of Renin-Angiotensin System and its receptors angiotensin receptor 1 (AT1) and 2 (AT2). Therefore, the role of angiotensin II in the pathology of cerebral malaria was investigated. To this end, it was observed that the disruption of interendothelial junctions and detachment caused by rupture and release of iRBCs over HBMECs is partially inhibited (only 41% damage compared to control) in the presence of Losartan, an AT1 antagonist (FIGS. 1C, 1E and 1F). Even greater inhibition of iRBC induced damage to HBMECs was observed in the presence of CGP-42112A (only 20% damage), an AT2 agonist (FIGS. 1D, 1E and 1F).

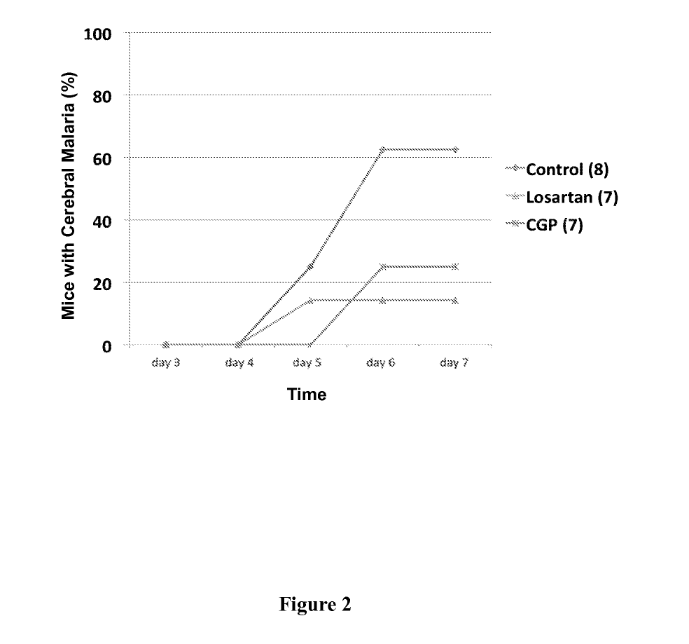

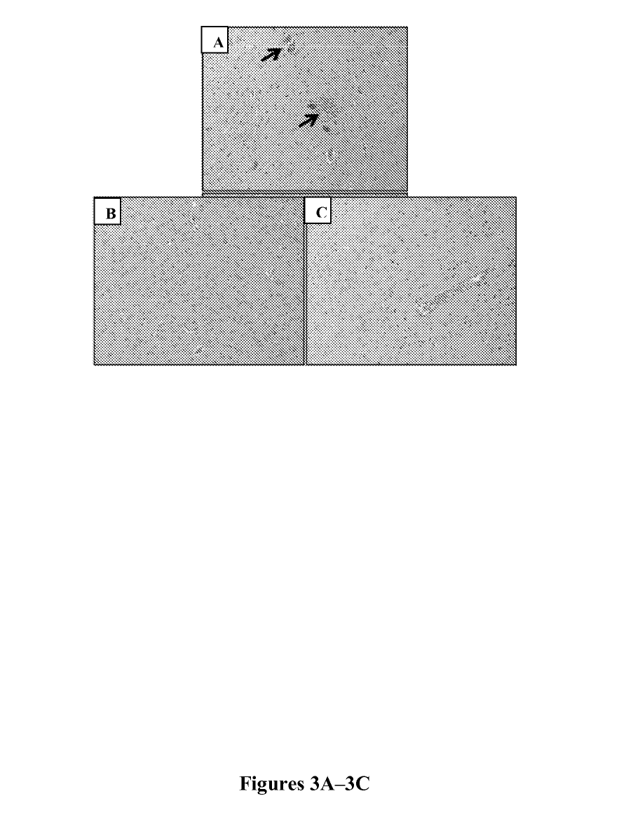

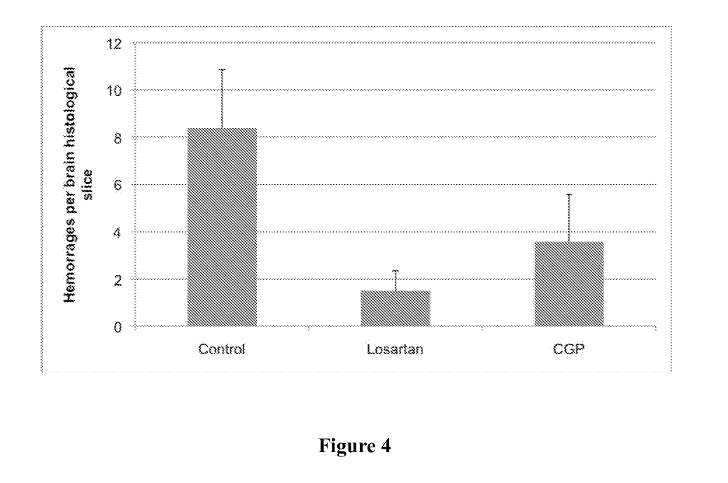

Furthermore, in an experimental mouse model of cerebral malaria, both the development of cerebral malaria and mortality were reduced by 4-5 times in mice receiving Losartan (n=7) compared with those animals with vehicle treatment (n=8) (FIG. 2). A similar beneficial effect was observed in mice administered CGP-42112A (n=7), an agonist of AT2. When the brains of these animals were analyzed, petechias and hemorrhages were found in the untreated groups, indicating the disruption of the blood brain barrier. In contrast, the structure of the brain vessels and the integrity of the blood brain barrier (BBB) was better maintained in animals receiving Losartan or CGP-42112A (FIGS. 3A-3C). Quantification of hemorrhages in brain histological slices shows the protective effect of Losartan and CGP-42112A (FIG. 4). Taken together, these results indicate that the balance between AT1 and AT2 receptors determines the outcome of cerebral malaria.

In summary, this Example demonstrates in human cells in vitro and in mouse experimental models, that antagonists of AT1 receptor and agonists of AT2 receptor prevent and/or inhibit the brain endothelial cells damage induced by Plasmodium and can be used for treatment and prevention of cerebral malaria

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. A pharmaceutical composition for the treatment or prevention of cerebral malaria, said pharmaceutical composition comprising:
an angiotensin receptor type-2 agonist; and
an antimalaria drug.

2. The composition of claim 1, wherein the angiotensin receptor type-2 agonist is selected from the group consisting of CGP-42112A, Compound 21, Novokinin, p-aminophenylalanine-angiotensin II, and combinations thereof.

3. The composition of claim 1, wherein the antimalaria drug is selected from the group consisting of quinine and derivatives thereof, chloroquine, amodiaquine, pyrimethamine, proguanil, sulfonamide, mefloquine, atovaquone, primaquine, artemisinin, artemether, artesunate, arteether, halofantrine, doxycycline, clindamycin, and combinations thereof.

4. The composition of claim 1 further comprising:
an angiotensin receptor type-1 antagonist.

5. The composition of claim 4, wherein the angiotensin receptor type-1 antagonist is selected from the group consisting of Losartan, Candesartan, Valsartan, Irbesartan, Telmisartan, Eprosartan, Olmesartan, Azilsartan, EXP3174, and combinations thereof.

6. The composition of claim 1 further comprising:
a pharmaceutically acceptable carrier.

7. The composition of claim 6, wherein said composition is formulated for oral, rectal, intravenous, intramuscular, intraperitoneal, or nasogastric tube administration.

8. A method of preventing or treating cerebral malaria in a subject, said method comprising:
selecting a subject having or at risk of having malaria and
administering, to the selected subject, an angiotensin receptor type-2 agonist under conditions effective to prevent or treat cerebral malaria in the subject.

9. The method of claim 8, wherein the angiotensin receptor type-2 agonist is selected from the group consisting of CGP-42112A, Compound 21, Novokinin, p-aminophenylalanine-angiotensin II, and combinations thereof.

10. The method of claim 8 further comprising:
administering an angiotensin receptor type-1 antagonist to the selected subject in conjunction with said administering the angiotensin receptor type-2 agonist.

11. The method of claim 10, wherein the angiotensin receptor type-1 antagonist is selected from the group consisting of Losartan, Candesartan, Valsartan, Irbesartan, Telmisartan, Eprosartan, Olmesartan, Azilsartan, EXP3174, and combinations thereof.

12. The method of claim 10, wherein the angiotensin receptor type-2 agonist and angiotensin receptor type-1 antagonist are administered simultaneously.

13. The method of claim 10, wherein the angiotensin receptor type-2 agonist and angiotensin receptor type-1 antagonist are administered sequentially.

14. The method of claim 8 further comprising:
administering one or more anti-malaria drugs to the subject in conjunction with said administering the angiotensin receptor type-2 agonist.

15. The method of claim 14, wherein the angiotensin receptor type-2 agonist and the anti-malaria drug are administered simultaneously.

16. The method of claim 14, wherein the angiotensin receptor type-2 agonist and the anti-malaria drug are administered sequentially.

17. The method of claim 14, wherein the antimalaria drug is selected from the group consisting of quinine and derivatives thereof, chloroquine, amodiaquine, pyrimethamine, proguanil, sulfonamide, mefloquine, atovaquone, primaquine, artemisinin, artemether, artesunate, arteether, halofantrine, doxycycline, clindamycin, and combinations thereof.

18. The method of claim 8, wherein the subject is a mammal.

19. The method of claim 18, wherein the mammal is a human.

20. The method of claim 8, wherein cerebral malaria is prevented in the subject.

21. The method of claim 8, wherein cerebral malaria is treated in the subject.

22. A method of preventing or treating cerebral malaria in a subject comprising:
selecting a subject having or at risk of having malaria and
administering, to the selected subject, an angiotensin receptor type-1 antagonist under conditions effective to prevent or treat cerebral malaria in the subject.

23. The method of claim 22, wherein the angiotensin receptor type-1 antagonist is selected from the group consisting of Losartan, Candesartan, Valsartan, Irbesartan, Telmisartan, Eprosartan, Olmesartan, Azilsartan, EXP3174, and combinations thereof.

24. The method of claim 22 further comprising:
administering an angiotensin receptor type-2 agonist and one or more anti-malaria drugs to the selected subject in conjunction with said administering the angiotensin receptor type-1 antagonist.

25. The method of claim 24, wherein the angiotensin receptor type-2 agonist is selected from the group consisting of CGP-42112A, Compound 21, Novokinin, p-aminophenylalanine-angiotensin II and combinations thereof.

26. The method of claim 24, wherein the one or more anti-malaria drugs is selected from the group consisting of quinine and derivatives thereof, chloroquine, amodiaquine, pyrimethamine, proguanil, sulfonamide, mefloquine, atovaquone, primaquine, artemisinin, artemether, artesunate, arteether, halofantrine, doxycycline, clindamycin, and combinations thereof.

27. The method of claim 24, wherein the angiotensin receptor type-1 antagonist, the angiotensin receptor type-2 agonist, the one or more anti-malaria drugs are administered simultaneously.

28. The method of claim 24, wherein the angiotensin receptor type-1 antagonist, the angiotensin receptor type-2 agonist, and the one or more anti-malaria drugs are administered sequentially.

29. The method of claim 22, wherein the subject is a mammal.

30. The method of claim 22, wherein the mammal is a human.

31. The method of claim 22, wherein cerebral malaria is prevented in the subject.

32. The method of claim 22, wherein cerebral malaria is treated in the subject.
